(19) United States
(12) Patent Application Publication
(10) Pub. No.: US 2005/0067514 A1
(43) Pub. Date: Mar. 31, 2005

(54) Freshwater supply fitting for water-conducting household appliances

(75) Inventors: Edwin Bolduan, Berlin (DE); Mauro Mazzo, Casatenovo (IT); Horst Wiemer, Kleinmachnow (DE)

Correspondence Address: LERNER AND GREENBERG, PA, P O BOX 2480, HOLLYWOOD, FL, 33022-2480, US

(73) Assignee: BSH Bosch und Siemens Hausgerate GmbH

(21) Appl. No.: 10/912,387

(22) Filed: Aug. 2, 2004

(30) Foreign Application Priority Data
Aug. 1, 2003 (DE) 103 35 330.5

(51) Int. Cl. A62B 7/00; A61M 16/00; B05B 15/08

(52) U.S. Cl. 239/600; 239/587.1

(57) ABSTRACT

In a connection between a freshwater supply tube and the connection device, in order to avoid parts other than the parts formed by the freshwater supply tube and the connection device themselves, and in order to simplify the connecting process compared with the prior art, a supply tube is provided and contains a corrugated tube made of a plastic (PE, PA or PP) suitable for maintaining drinking water quality. An end of the corrugated tube has a substantially cylindrical shape and is coupled to the connection device. The corrugated tube is covered on the outer side with a fabric sheath of polyamide threads or fibers. The connection device is connected to the corrugated tube in a captive manner by encapsulating the cylindrical end by the plastic of the connection nozzle.

### BACKGROUND OF THE INVENTION

1. Field of the Invention

The invention relates to a freshwater supply fitting for water-conducting household appliances. The supply fitting has a connection device, attached to at least one end and intended for connection to a freshwater fixed network, and a flexible corrugated supply tube.

The corrugated supply tube is at least approximately cylindrically shaped at its end and is connected to a matching cylindrical connection nozzle of a connection device by the cylindrical end being drawn over the connection nozzle and being connected in a captive manner by a press sleeve. When the corrugated tube is covered with a nylon fabric, the fabric structure certainly presses into the surface of the cylindrical end, but effective locking and reliable sealing are nonetheless only achieved when an annular structure in which the remaining parts are molded in place during the pressing is provided on the connection nozzle and if an O-ring-like seal is also inserted between the cylindrical end and the connection nozzle.

2. Summary of the Invention

It is accordingly an object of the invention to provide a freshwater supply fitting for water-conducting household appliances that overcomes the above-mentioned disadvantages of the prior art devices of this general type, in which no parts other than the freshwater supply tube and the connection device itself are required for connecting the freshwater supply fitting together and in which the connecting process is simpler than in the prior art.

With the foregoing and other objects in view there is provided, in accordance with the invention, a freshwater supply fitting for a water-conducting household appliance. The freshwater supply fitting contains a connection device disposed at one end and provided for connecting to a freshwater fixed network. The connection device has a connection nozzle formed of plastic. A flexible corrugated supply tube contains a corrugated tube made of a plastic suitable for maintaining drinking water quality. The corrugated tube has a cylindrical end coupled to the connection device and is at least substantially cylindrically shaped. The connection device is connected to the corrugated tube in a captive manner by encapsulating the cylindrical end by the plastic of the connection nozzle. A fabric sheath covering is disposed on an outer side of the cylindrical end of the corrugated tube. The fabric sheath is formed of polyamide threads or fibers.

According to the invention, the supply tube contains a corrugated tube that can be made of plastic (PE, PA or PP) that maintains drinking water quality. The end of the corrugated tube that is coupled to the connection device is at least approximately cylindrically shaped and is covered on the outside with a fabric sheath of polyamide threads or fibers. The connection device is connected to the corrugated tube in a captive manner by encapsulating the cylindrical end by the plastic of the connection nozzle. Due to the invention, it is possible for the first time to use plastics suitable for drinking water for freshwater connection fittings that can be tightly and firmly connected to one another in the simplest manner.

In a configuration of this solution according to the invention, the connection nozzle is of a double-walled configuration over a restricted length from its free end, and the cylindrical end is inserted between the walls of the double-walled section and is connected to the double-walled section by latching, adhesive bonding, welding or injection molding. This results in an especially fluidically favorable internal shaping for the connection region and at the same time the resistance of the tube against being torn off the connection device is especially high.

A further alternative to the solution of the object includes the supply tube containing the corrugated tube made of plastic (PE, PA or PP) maintaining drinking water quality. The end of the corrugated tube that is coupled to the connection device is at least approximately cylindrically shaped and is connected between two parts of a connection nozzle, produced from plastic, of the connection device by a clamping connection of the tube end. These two parts are connected to one another by latching, adhesive bonding, welding or injection molding. The same advantages as for the first alternative apply to this.

In an especially advantageous development of the second alternative of the freshwater supply fitting, the end of the corrugated tube has a bent-up collar at its orifice, this collar lying between outer walls, running radially relative to the center line of the connection nozzle, of the two parts. As a result, the resistance of the tube against being torn off the connection device is especially high.

Other features which are considered as characteristic for the invention are set forth in the appended claims.

Although the invention is illustrated and described herein as embodied in a freshwater supply fitting for water-conducting household appliances, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE DRAWINGS

FIG. 1 is a diagrammatic, sectional view of a supply fitting in which a cylindrical end of the supply tube is encapsulated by a connection nozzle of a connection device according to the invention;

FIG. 2 is a diagrammatic, sectional view of the supply fitting, in which the cylindrical end is pushed into the cavity of a double-walled section of the connection nozzle according to a second exemplary embodiment of the invention;

FIG. 3 is a diagrammatic, sectional view shows of a third exemplary embodiment of the invention, in which the cylindrical end of the supply tube is provided with a collar, which is held in a form-locking manner between two parts of the connection device; and

FIG. 4 is a diagrammatic, sectional view of a fourth exemplary embodiment similar to that of FIG. 3.

### DESCRIPTION OF THE PREFERRED EMBODIMENTS

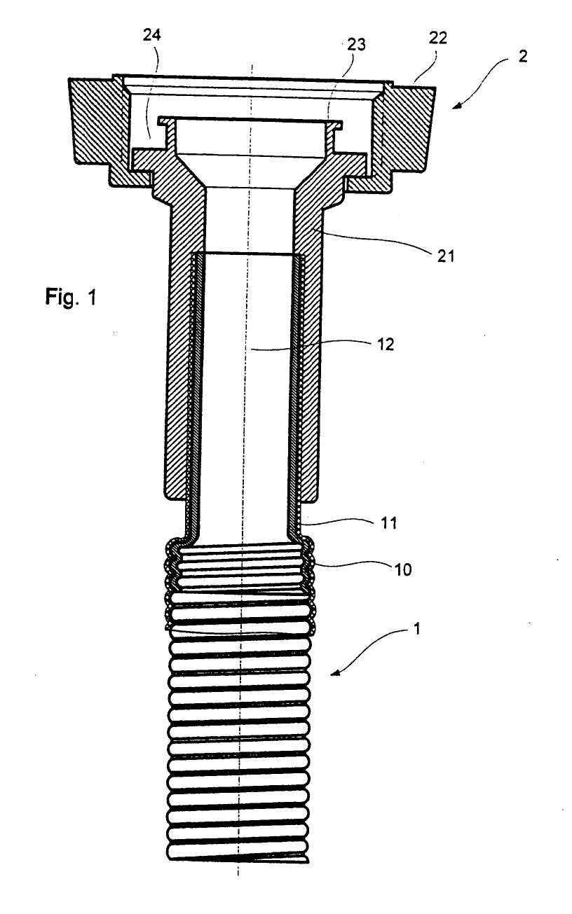

Referring now to the figures of the drawing in detail and first, particularly, to FIG. 1 thereof, there is shown a supply tube 1 formed of a corrugated plastic tube 1 made of a plastic suitable for drinking water, namely PE, PA or PP, and of a fabric tube 10 drawn over the plastic tube 11 and made of NYLON. According to FIG. 1, the fabric tube 10 is drawn right into a cylindrically shaped end 12 of the corrugated tube 11. The cylindrical end 12 of the corrugated tube 11 can be given a sufficiently dimensionally accurate cylindrical shape by a thermal method in an especially simple manner, to be precise both when producing corrugated tube sections in finished lengths and in the case of tubes in meter lengths, in which the cylindrical shape for the end section of the corrugated tube can be reshaped and can serve in this form as a connection part for connecting to the connection device.

A connection device 2 may be formed of a tubular connection nozzle 21 and a cap nut 22, by which the supply fitting can be fastened to a non-illustrated threaded connection part.

Before the connection is produced, the end 12 of the corrugated tube 11 is reshaped into a cylindrical part and the entire tube is covered with the strength-increasing fabric tube 10. The cylindrically shaped end 12 of the supply tube 1 is then inserted into an injection mold and is encapsulated with a plastic, e.g. PA, for the connection nozzle 21. In the process, the plastic of the connection nozzle 21 forms an intimate connection with the fabric tube 10. The connection between the connection device 2 and the supply tube 1 is thus complete.

Inside a cap nut 22, the connection nozzle 21 has an extension 23 which, on its outside, forms together with the nut 22 an annular space 24 for a non-illustrated sealing ring which serves for the sealing connection between the connection device 2 and a non-illustrated threaded part, which may be an integral part of a valve of a domestic water network.

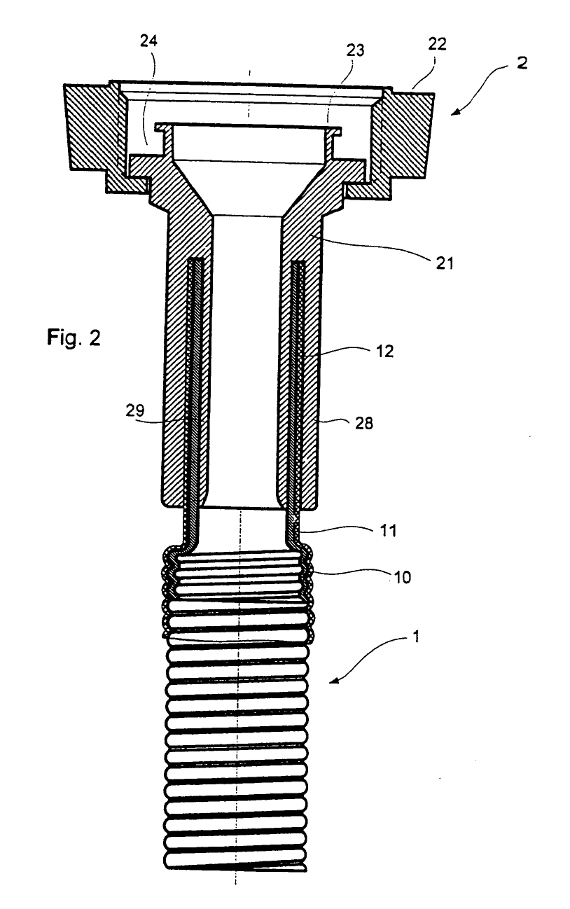

In the variant shown in FIG. 2, the cylindrical end 12 of the supply tube 1 is pushed into an annular cavity of a double-walled section 28, 29 of the connection nozzle 21. The cylindrical end 12 can be connected therein to the double-walled section by latching or adhesive bonding or welding or injection molding. For example, the cylindrical end 12 can be completely encapsulated with the fabric tube by the plastic of the double-walled connection nozzle 21.

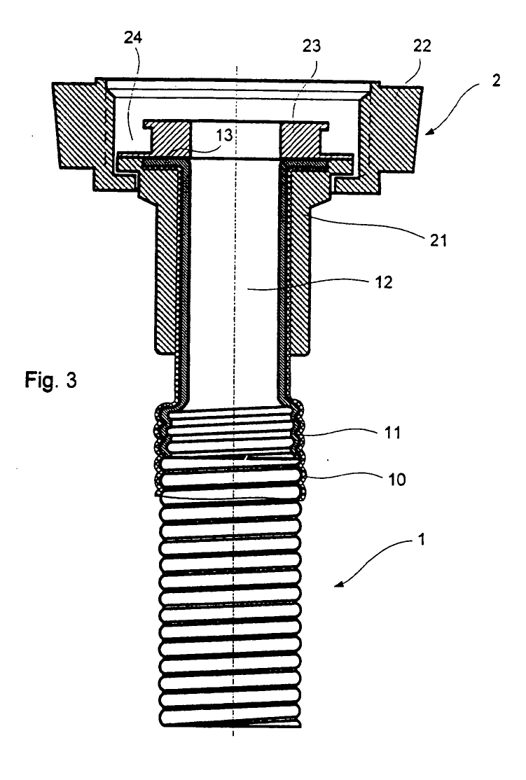

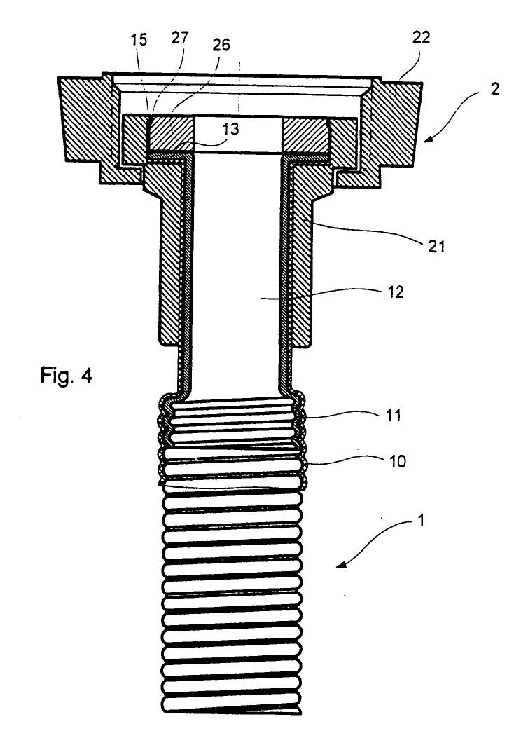

On the connection side, the cylindrical end 12 of the supply tube 1 according to the examples in FIGS. 3 and 4 is bent at right angles in a flange-like manner. In the example shown in FIG. 3, the outside of the flange 13 is flush with the end face of the cylindrical end 12, so that both are covered by a seal retaining ring 25 by its corresponding annular surface. Flush welding is the quickest way of producing the connection between the seal retaining ring 25 and the cylindrical end 12, on the one hand, and the end face of the cylindrical end 12, on the other hand. Other connecting techniques such as adhesive bonding, injection molding or latching are likewise possible.

FIG. 4 gives an example for a latched connection. The exemplary embodiment shown in FIG. 4 exhibits a hollow for the cylindrical connection nozzle 21, the hollow serving to accommodate the flange 13 bent at an angle, on the one hand, and a closure ring 26 on the other hand. At the front margin, the closure ring 26 has an annular notch 27, which interacts with at least three latching lugs 15, distributed over the inner circumference of the hollow, in such a way that the closure ring 26 is firmly held in the hollow as a result. The flange 13 bent at right angles is thus fastened in the hollow at the same time, so that the connection nozzle 21 is connected to the cylindrical end 12 of the supply tube 1 in a captive manner.

The contours shown for the connection device 2—insofar as they are not in direct contact with the cylindrical end 12—are not subject matter of the invention. Thus the cap nut 22 is not included in the scope of protection of the claims.

This application claims the priority, under 35 U.S.C. § 119, of German patent application No. 103 35 330.5, filed Aug. 1, 2003; the entire disclosure of the prior application is herewith incorporated by reference.

1. A freshwater supply fitting for a water-conducting household appliance, the freshwater supply fitting comprising:
a connection device disposed at one end and provided for connecting to a freshwater fixed network, said connection device having a connection nozzle formed of plastic;
a flexible corrugated supply tube containing a corrugated tube made of a plastic suitable for maintaining drinking water quality, said corrugated tube having a cylindrical end coupled to said connection device and being at least substantially cylindrically shaped, said connection device connected to said corrugated tube in a captive manner by encapsulating said cylindrical end by said plastic of said connection nozzle; and
a fabric sheath covering on an outer side of said cylindrical end of said corrugated tube, said fabric sheath formed of a material selected from the group consisting of polyamide threads and fibers.

2. The freshwater supply fitting according to claim 1, wherein said connection nozzle has a free end and a double-walled section with walls extending over a restricted length from said free end of said connection nozzle, said cylindrical end disposed between said walls of said double-walled section and is connected to said double-walled section by one of latching, adhesive bonding, welding and injection molding.

3. A freshwater supply fitting for a water-conducting household appliance, the freshwater supply fitting comprising:
a connection device disposed at one end and provided for connecting to a freshwater fixed network, said connection device having a connection nozzle and a fixing part both formed from plastic;
a flexible corrugated supply tube containing a corrugated tube made of plastic suitable for maintaining drinking water quality, said corrugated tube having a cylindrical end with a substantially cylindrical shape, said cylindrical end of said corrugated tube coupled to said connection device by being clamped between said fixing part and said connection nozzle, said fixing part and said connection nozzle being connected to one another by one of latching, adhesive bonding, welding and injection molding; and
a fabric sheath covering an outer side of said corrugated tube, said fabric sheath made of a material selected from the group consisting of polyamide threads and fibers.

4. The supply fitting according to claim 3, wherein said cylindrical end of said corrugated tube has an orifice formed therein and a bent-up collar disposed at said orifice, said bent-up collar lying between outer walls of said fixing part and said connection nozzle and runs radially relative to a center line of said connection nozzle.
